(19) United States
(12) Patent Application Publication
(10) Pub. No.: US 2016/0067413 A1
(43) Pub. Date: Mar. 10, 2016

(54) SYRINGES

(71) Applicant: STAR SYRINGE LIMITED, London (GB)

(72) Inventors: Graham John Madin, Mielkendorf (DE); Paul Mallins, Verbier (CH); David Michael Bower, West Sussex (GB)

(21) Appl. No.: 14/787,437

(22) PCT Filed: Apr. 23, 2014
(86) PCT No.: PCT/GB2014/051256

§ 371 Date: Oct. 27, 2015

(30) Foreign Application Priority Data
Apr. 24, 2013 (GB) 1307385.3

(51) Int. Cl. A61M 5/315; A61M 5/32; A61M 5/34

(52) U.S. Cl. CPC A61M 5/31513; A61M 5/344; A61M 5/3202

(57) ABSTRACT

A syringe 1 comprises a barrel 2 and a plunger 3 sliding in the barrel and having a seal 8 in sealing engagement with an internal surface of the barrel, the barrel having a piercing member 20 with a piercing point 27 and an aperture 26 at its distal end. The aperture 26 is in communication with the distal end of the barrel, and the distal end of the barrel has a connection member 28 to which an adapter member 36 provided with an intradermal needle 39 is adapted to be attached. An intradermal injection adapter 36 for a syringe is also provided, the adapter comprising a body 37 having an intradermal needle 39 at a distal end, and a proximal end constructed and arranged for attachment to the barrel, the body also providing a fluid connection between the barrel 2 and the intradermal needle 39.

This invention relates to syringes, and in particular to intradermal syringes.

Intradermal injection is defined as injection into the epidermis and upper layers of the dermis, typically to a depth of 2-3 mm, before the subcutaneous tissue and muscle tissue are reached. It is currently used for the administration of specific vaccines, BCG and rabies, that would be dangerous if not delivered intradermally. Intradermal injection with a standard needle and syringe is performed using the Mantoux technique. The Mantoux technique is recognised as being difficult, and requires a high level of skill to be successful if a standard needle and syringe is to be used to give an intradermal injection to a depth of 2-3 mm, rather than an intramuscular injection to a depth of about 1 cm. The skin is stretched, the needle is inserted at an angle of about 15° to the skin (instead of perpendicularly), and a small volume of liquid injected. If the injection is successful, a hemispherical blister (known as a bleb) is formed under the surface of the skin. As a result, most vaccines are currently delivered by intramuscular or subcutaneous injection using a standard needle and syringe. Because the skin layers formed by the epidermis and dermis are good at absorbing and distributing antigens, delivery of vaccines to these layers, rather than to muscle or subcutaneous tissue, should be more efficient and induce protective immune responses with smaller amounts of vaccine. As a result, smaller doses of vaccine can be given than if the vaccine is delivered by a standard intramuscular injection. Typically, an intradermal injection might require only 0.05 ml to 0.1 ml of vaccine, rather than 0.5 ml for an intramuscular injection. The reduced dosage, known as dose-sparing, has obvious advantages, especially in immunisation programmes in developing countries, as more doses can be obtained from the existing vaccine supply. This reduces the cost (including transport and storage) of each injection and means that many more people can be immunised for the same cost. It could be similarly advantageous if mass immunisation against influenza, for example, is required because of a possible pandemic (when there is never sufficient antigen to meet global demand). Of course, intradermal injection may also be used to deliver other diagnostics, therapies or experimental applications, and will have similar advantages in dose reduction.

Because the Mantoux technique is difficult, devices designed for intradermal injection have been developed. These may use micro needles or nano needles, jet injectors or patches. Some devices use a very fine needle arranged so that it can be inserted perpendicularly to the skin, but with its penetration limited to a maximum of 3 mm. It is preferable to ensure that the needle is protected after use to reduce the risk of needlestick injury, and that the device cannot be re-used. One difficulty is that the very short intradermal needle is not able to draw up the liquid injectant from a vial, as it cannot penetrate deep enough into a vial or ampoule to draw the liquid into the barrel of the device. Also only auto-disable devices are approved for developing countries. With all these requirements, the devices may be complex, and therefore difficult to provide for use in developing countries. For example, the device shown in WO 2002/083212 has a prefillable reservoir with a plunger, the fine needle being attached to the reservoir. The reservoir is located inside a limiter and a sleeve, and the needle end sealed by a pierceable elastomeric insert. For use, the plunger is depressed. Its initial movement forces the needle through the elastomeric insert to project by the required amount, determined by the engagement of the reservoir with the limiter. Further movement then delivers the injectant. A spring acts to return the reservoir, retracting the needle into the limiter and locking the limiter to the sleeve to prevent re-use of the device. While this device delivers the injectant at the correct depth, protects the needle after use and prevents re-use, it is not suitable for use in a situation where the vaccine or any other liquid must be drawn up from a vial or ampoule just before use, as required in developing countries, because the reservoir must be pre-filled.

According to a first aspect of the present invention, we provide a syringe comprising a barrel and a plunger sliding in the barrel and having a seal in sealing engagement with an internal surface of the barrel, the barrel having a piercing member with a piercing point and aperture at its distal end, the aperture being in communication with the distal end of the barrel, the distal end of the barrel also having a connection member to which an adapter member provided with an intradermal needle is adapted to be attached.

The piercing member is used to draw up injectant from a vial or ampoule, and the adapter member is then attached for intradermal injection, through the piercing member and the intradermal needle. The construction of the syringe is simple, and it can be used to deliver an intradermal injection in the situation where the injectant must be drawn up from a vial. This makes it able to be used with any sized vial or ampoule thus enabling filling at the point of use, which is a pre-requisite in developing countries, and its simple construction means that it is relatively less expensive to produce. It is also easy to use, requiring only limited training of health care workers.

The piercing member and the connection member are conveniently formed integrally with the barrel. This has the advantage of simplicity.

The piercing point at the distal end of the piercing member is preferably formed by an angled end of the piercing member, which also provides the aperture. The aperture may lead to a bore in the piercing member which provides communication with the distal end of the barrel. The piercing member and the bore are conveniently tapered towards the point to assist with fluid flow. Alternatively, the aperture may lead directly to the distal end of the barrel.

The connection member may comprise a collar member surrounding the piercing member or the distal end of the barrel. An annular gap is provided between the piercing member or distal end of the barrel and the collar member to accommodate the adapter member. The collar member is arranged to lock the adapter member in place. This prevents removal of the adapter member and thus its re-use. The annular gap also accommodates a transport cap, which is assembled with the barrel to protect the piercing member for storage and transport until the syringe is to be used.

The adapter member conveniently comprises a body having at its proximal end a locking means for cooperation with the collar member, and carrying the intradermal needle at its distal end. The distal end may be arranged to accommodate a closure member to protect the intradermal needle before and after use.

The adapter body has an internal recess at its proximal end to accommodate the piercing member. The recess is tapered to correspond to the external profile of the piercing member. At its distal end the recess leads to a narrow axial bore in which the intradermal needle is accommodated. The needle projects beyond the distal end of the adapter body by a small amount. Conveniently this may be 1.5 mm. Penetration of the needle into the skin is limited by the distal end of the body engaging with the skin surrounding the needle. The distal end of the adapter body is substantially flat, and perpendicular to the axial bore. It has a conical recess through which the needle projects, and an annular recess spaced from and surrounding the conical recess, allowing space for the bleb to form following an injection.

The closure member for the distal end of the adapter body conveniently comprises a cap hinged to the adapter body. The hinge may be formed integrally with the body, the arrangement being such that the cap may be flipped off for injection and flipped back on after use with the hand of the user always behind the needle. This reduces the risk of a needlestick injury after use. The proximal end of the cap has a conical recess to accommodate the projecting part of the needle, and an annular projection corresponding to the annular recess in the distal end of the adapter body. With the cap closing the adapter body, the projection is located in the recess, to assist in securing the cap in place.

At its proximal end the outer surface of the adapter body is shaped for attachment to the connection member. The adapter body has at least one projection adapted to engage in a corresponding aperture in the connection member. Conveniently a pair of opposing projections and apertures is provided. Each projection has a snap engagement with its corresponding aperture, the arrangement being a locking engagement such that the adapter body cannot be removed. The connection member may have a tapered surface leading to each aperture to guide the corresponding projection to the correct place. This ensures that each projection engages correctly, and controls the resilient expansion of the connection member to ensure the locking engagement.

The barrel may be of stepped outline, with a proximal portion of standard diameter and a distal portion of reduced diameter. The seal of the plunger is at the distal end, so that the seal engages in the reduced diameter portion of the barrel. This provides that where a small dose of injectant is required, it will be accommodated in the reduced diameter portion. It results in a larger plunger travel distance and increased accuracy of dosage volume. This has the advantage of reducing the dead space (the volume of injectant remaining in the syringe after use) so that injectant is not wasted. Where the aperture in the piercing member leads directly to the reduced diameter portion of the barrel, rather than the bore, the dead space is minimised. The plunger preferably has an auto-disable mechanism provided in the proximal portion of the barrel. This ensures that the syringe cannot be re-used, and as described above the adapter body also cannot be removed for re-use.

According to a second aspect of the present invention we provide an intradermal injection adapter for a syringe, the syringe comprising a barrel and a plunger in slidable and sealing engagement therein, and the adapter comprising a body having an intradermal needle at a distal end, and a proximal end constructed and arranged for attachment to the barrel, the body also providing a fluid connection between the barrel and the intradermal needle.

The adapter enables a standard syringe to deliver an intradermal injection without needing to use the Mantoux technique described above, because the intradermal needle enables the injection to be given with the syringe perpendicular to the skin surface. It can also provide a simple way of performing an intradermal injection using a pre-filled syringe.

The adapter may have a standard luer connection to the syringe barrel, being attached instead of a standard needle to a hub of the barrel. Instead, the adapter may have a locking connection to the barrel.

Conveniently the adapter has a closure member able to be attached at its distal end to protect the intradermal needle before and after use. The closure member may be hinged to the adapter body.

The distal end of the adapter body and the closure member may also have the features described above in relation to the adapter body of the first aspect of the invention.

Embodiments of both aspects of the invention are illustrated, by way of example only, in the accompanying drawings, in which:—

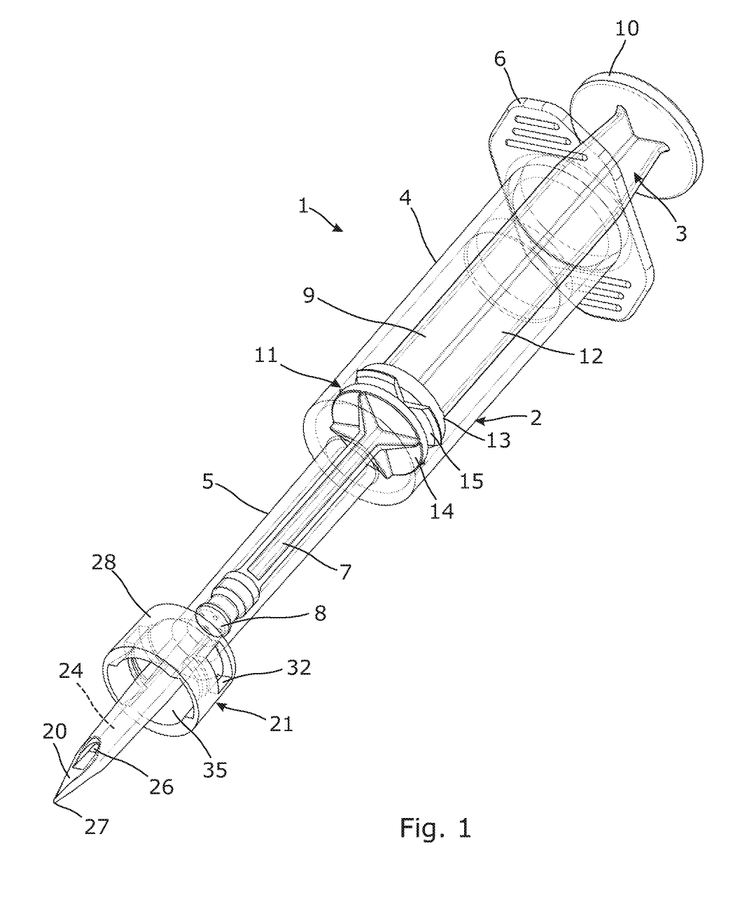

FIG. 1 is a perspective view of a syringe according to the first aspect of the invention;

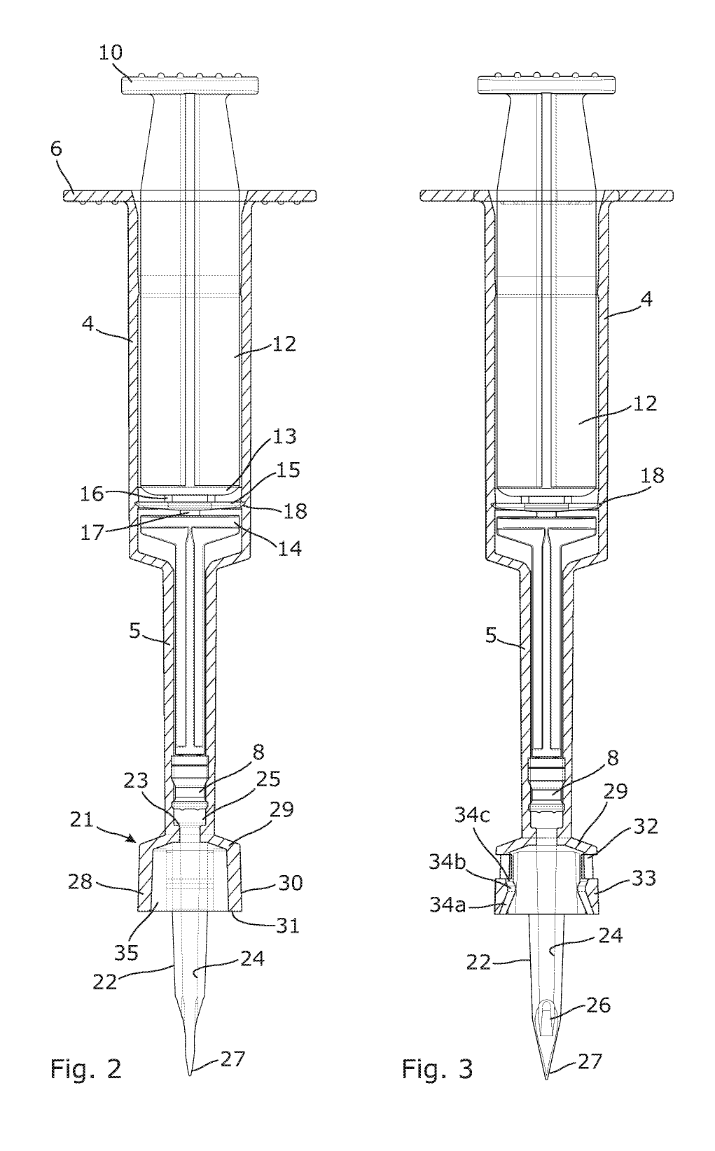

FIG. 2 is an axial cross-section through the syringe of FIG. 1;

FIG. 3 is similar to FIG. 2, but with the cross-section taken in a plane at right angles;

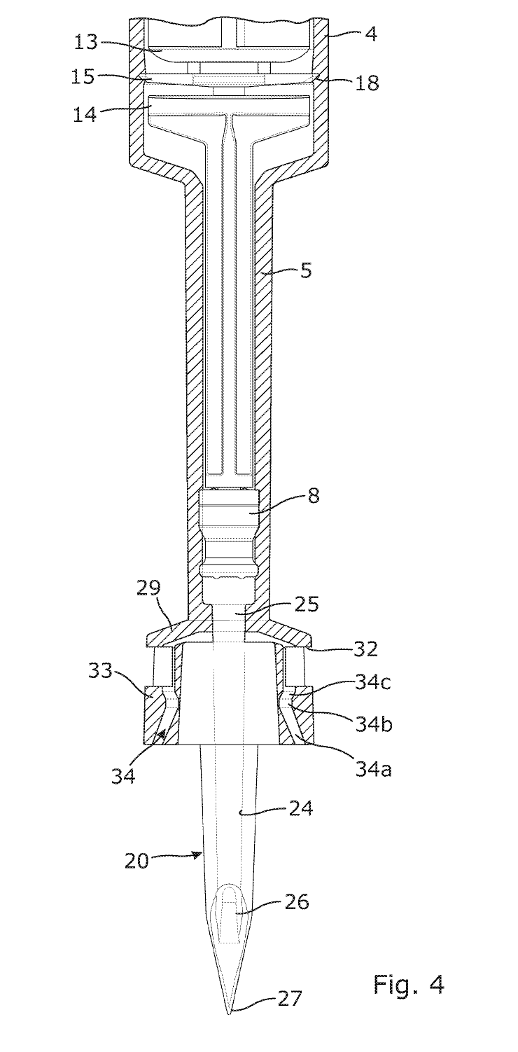

FIG. 4 is an enlarged view of part of the syringe of FIG. 2;

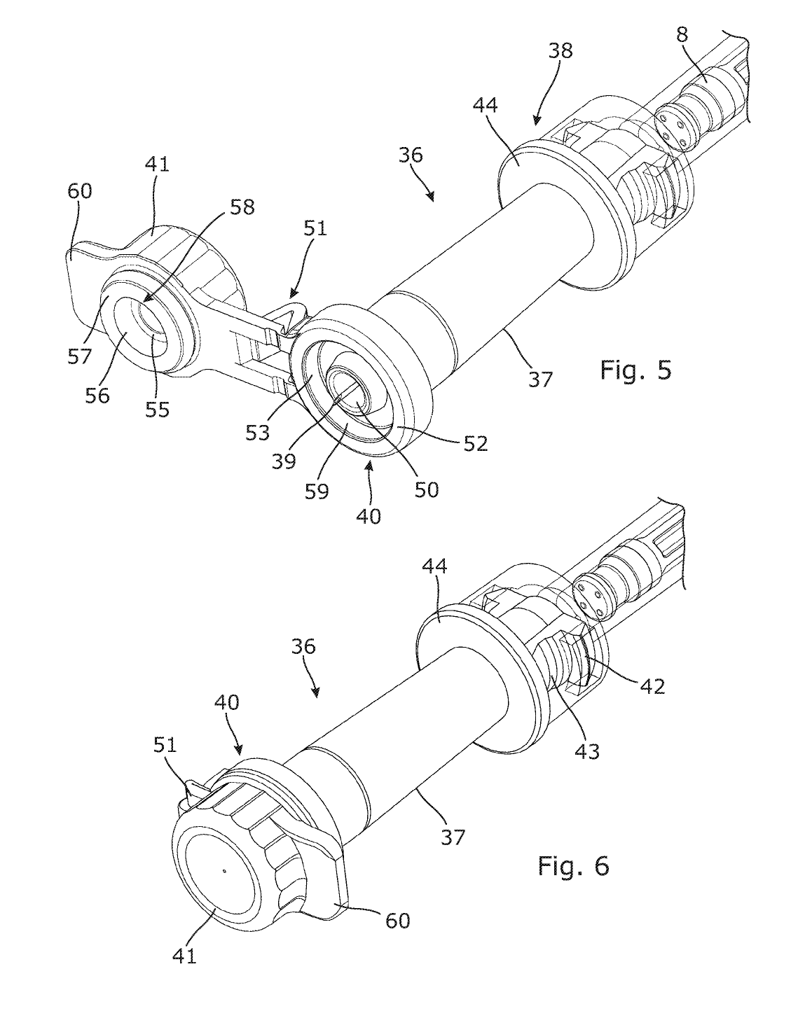

FIG. 5 is a perspective view of an intradermal adapter for the syringe of FIGS. 1 to 6;

FIG. 6 shows the adapter of FIG. 5 in a closed position;

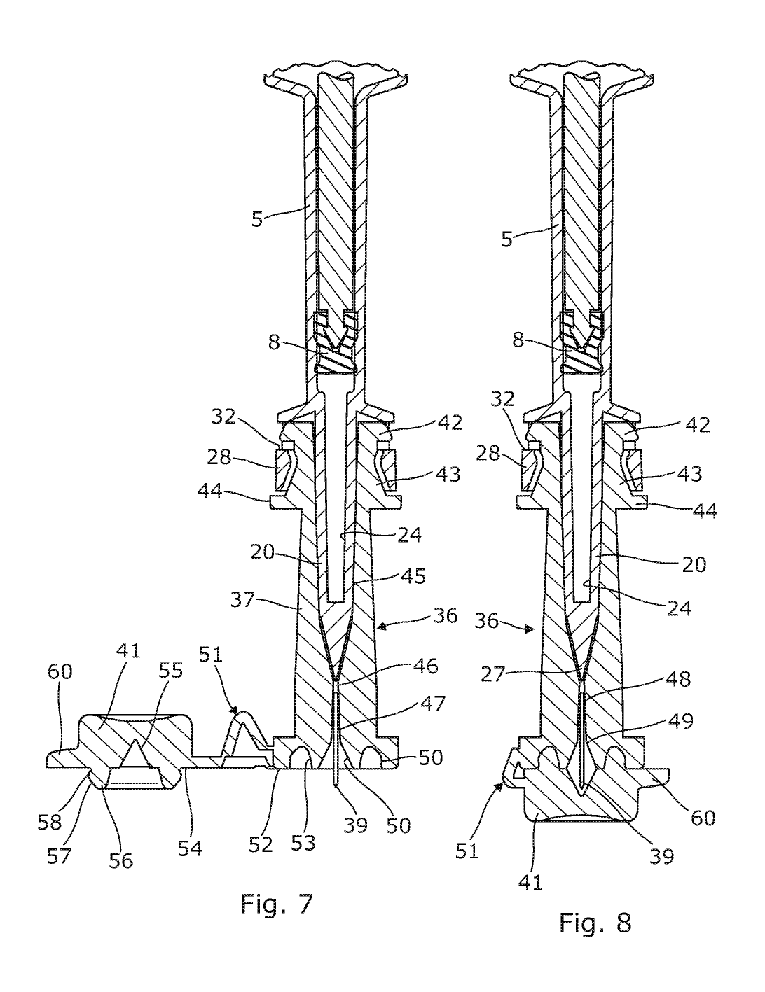

FIG. 7 is an axial cross-section through the syringe with the adapter in the open position;

FIG. 8 is similar to FIG. 7, but with the adapter in the closed position;

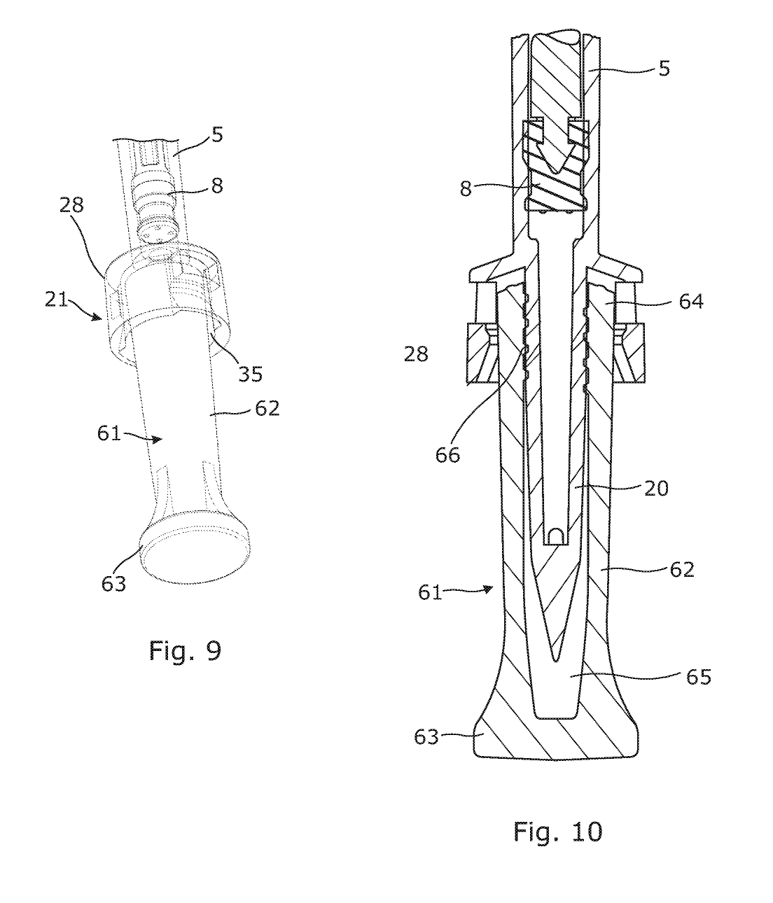

FIG. 9 is similar to FIG. 1, but includes a transport cap;

FIG. 10 is an axial cross-section through part of FIG. 9;

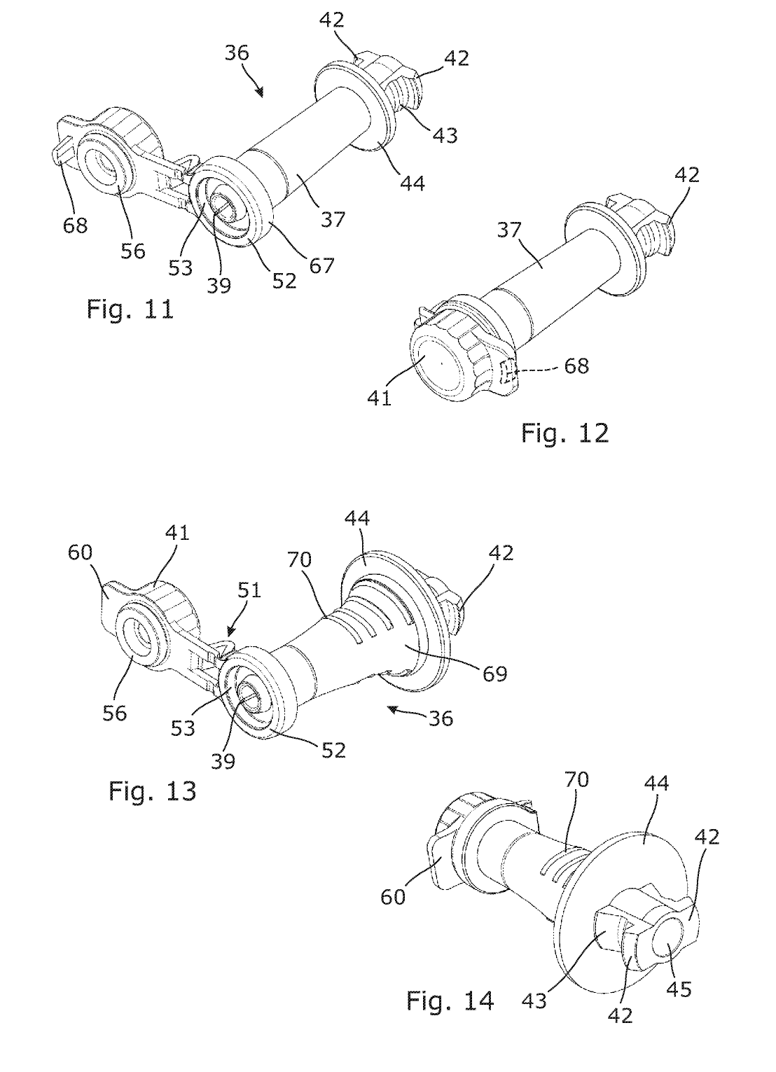

FIGS. 11 and 12 are perspective views of a modified adapter in the open and closed positions;

FIGS. 13 and 14 are perspective views of a second modified adapter in the open and closed positions, with FIG. 14 showing a reverse view;

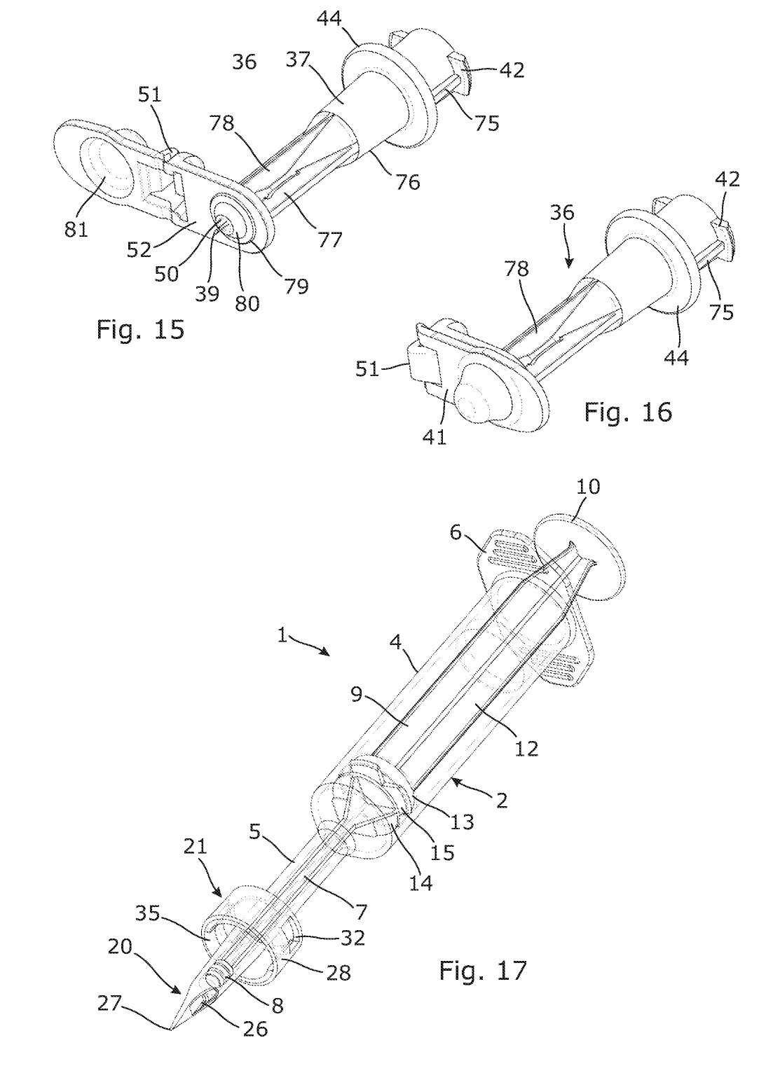

FIGS. 15 and 16 are perspective views of a further modified adapter in the open and closed positions;

FIG. 17 is a perspective view of a modified syringe according to the first aspect of the invention;

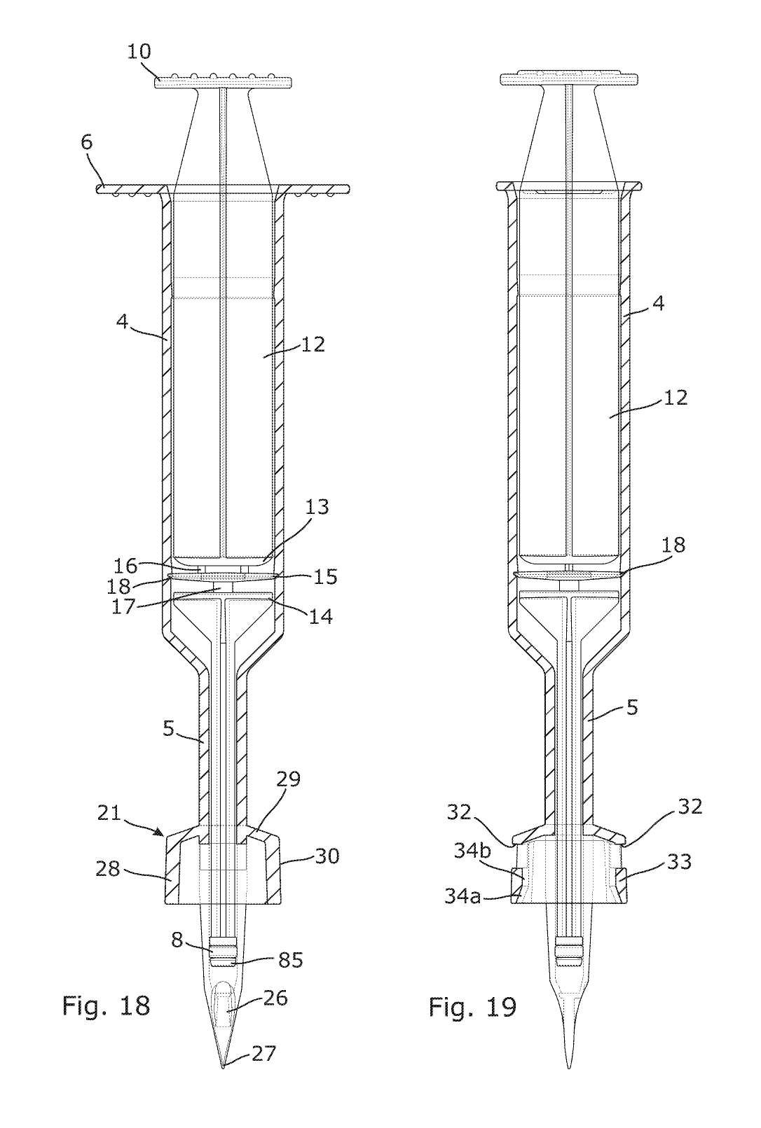

FIG. 18 is an axial cross-section through the syringe of FIG. 17;

FIG. 19 is similar to FIG. 18, but with the cross-section taken in a plane at right-angles;

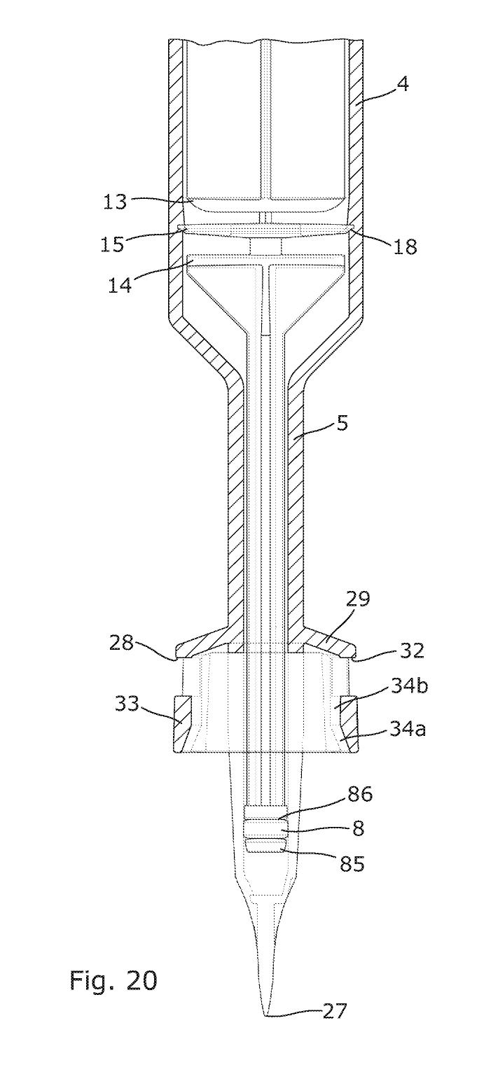

FIG. 20 is an enlarged view of part of the syringe of FIG. 18;

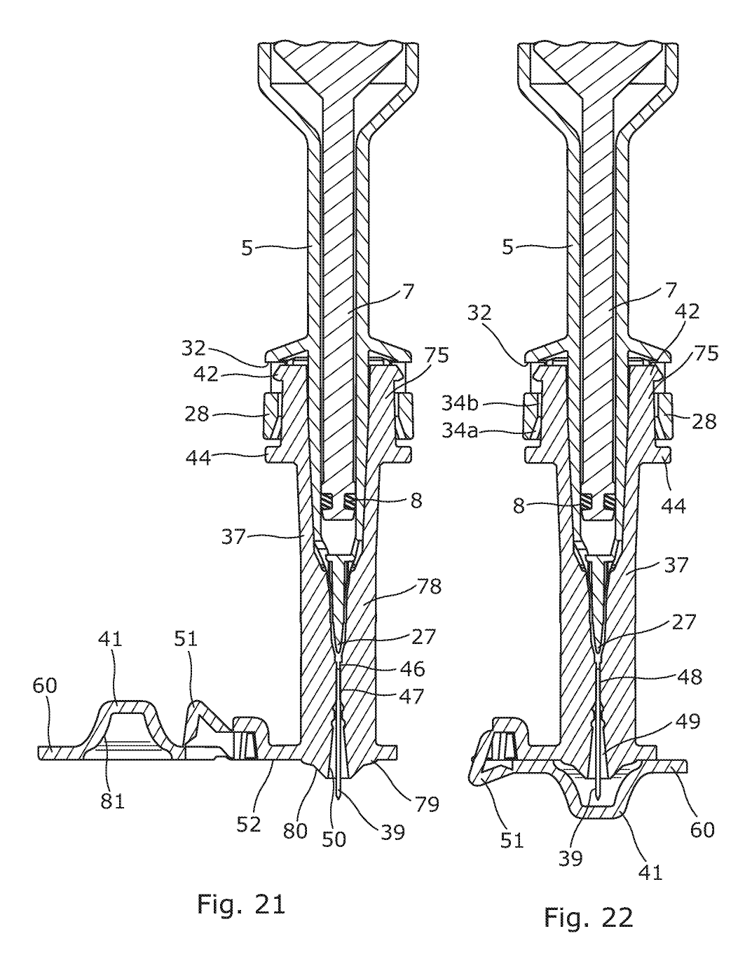

FIG. 21 is an axial cross-section through the syringe of FIG. 17 with the adapter of FIGS. 15 and 16 in the open position;

FIG. 22 is similar to FIG. 21, but with the adapter in the closed position;

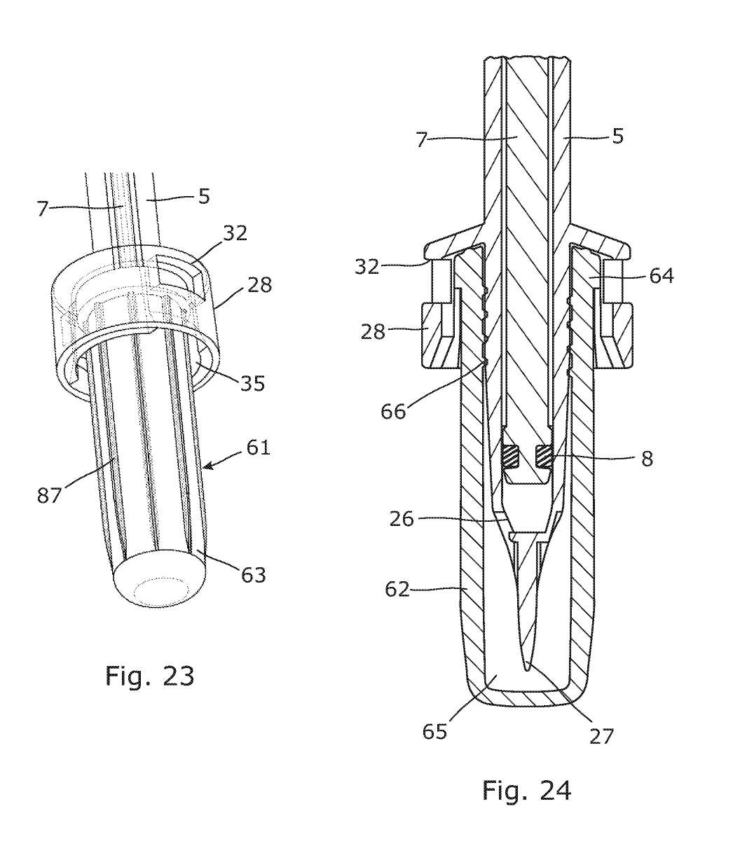

FIG. 23 is similar to FIG. 17, but includes a transport cap; and

FIG. 24 is an axial cross-section through part of FIG. 23.

FIGS. 1 to 3 show a syringe 1 designed for intradermal injection of a small amount of vaccine or other injectant. FIGS. 1 to 3 show the syringe 1 ready to draw up the injectant from a vial (not shown) and before the attachment of an intradermal needle for injection.

The syringe 1 comprises a cylindrical barrel 2 in which works a plunger 3. The barrel 2 is of stepped outline, having a proximal part 4 of larger diameter with a proximal open end and a distal part 5 of smaller diameter. A flange 6 is provided at the open end.

The plunger 3 has a distal rod 7 which carries an elastomeric head 8 in sealing engagement with the internal surface of the smaller diameter distal part 5, and a proximal portion 9 accommodated in the larger diameter proximal part 4. The proximal portion 9 comprises a proximal end disc 10 which is manually operable to move the plunger 3 in the barrel 2, an auto-disable mechanism 11 at the distal end, and a cruciform rod 12 connecting the disc 10 and the auto-disable mechanism 11. The auto-disable mechanism 11 comprises a pair of axially spaced discs 13, 14 with a cruciform member 15 between them. The proximal disc 13 is connected to the cruciform rod 12, and to the proximal side of the cruciform member 15 by two diametrally opposed thin stems 16. The distal disc 14 is connected to the distal side of the cruciform member 15 by a central stem 17. The distal side of the distal disc 14 is attached to the distal rod 7. The plunger 3 is injection moulded from plastics material as a unitary member.

As best seen in FIGS. 2 and 3, the barrel 2 has a groove 18 in its internal surface at the distal end of the proximal part 4. The cruciform member 15 is caught in the groove 18 when the plunger 3 is fully depressed, to prevent withdrawal of the plunger 3, and to cause operation of the auto-disable mechanism 11, as explained below.

The distal end of the barrel 2 is formed with a piercing member 20 and a connection member 21 for attachment of an adapter provided with an intradermal needle.

The piercing member 20 comprises a tapered cylindrical member 22 provided at the distal end of the smaller diameter distal part 5, such that a step 23 is formed between the distal part 5 and the cylindrical member 22. The cylindrical member 22 tapers distally, and has a correspondingly tapered internal open-ended bore 24. The proximal end 25 of the bore 24 is in communication with the distal part 5, and the distal end has an aperture 26 adjacent to a piercing point 27 formed at the distal end of the cylindrical member 22. The piercing point 27 is provided by forming the distal end at an angle, and is able to pierce a rubber septum of a vial (not shown) containing injectant. The injectant can be drawn up through the tapered bore 24 into the distal part 5 of the barrel 2 by withdrawing the plunger 3 manually in the usual way.

The connection member 21 comprises a collar 28 surrounding the proximal end of the tapered member 22, and is shown in more detail in FIG. 4. The collar 28 is cup-shaped, with a base 29 provided at the step 23, and a cylindrical wall 30 extending distally from the base 29. The cylindrical wall 30 is slightly conical, such that its open distal end 31 has a larger diameter than the base 29. The wall 30 has a pair of diametrally opposed rectangular apertures 32 adjacent the base 29. A wall portion 33 leading from the open end 31 to each aperture 32 has a tapered profile 34, comprising a relatively steep inwardly-angled entry part 34a, a short axially-extending part 34b, and a short outwardly-angled part 34c leading to the aperture 32. The collar 28 provides an annular space 35 surrounding the proximal end of the piercing member 20 to accommodate an adapter member, with the apertures 32 and wall portions 33 providing a locking engagement for the adapter member, as explained below.

Like the plunger 3, the barrel 2, including the piercing member 20 and connection member 21, can be moulded in one piece from plastics material.

The adapter member 36 is shown in FIGS. 5 to 8. It comprises a substantially cylindrical adapter body 37 arranged for attachment to the collar 28 at its proximal end 38 and carrying an intradermal needle 39 at its distal end 40, which also has a closure member 41. FIGS. 5 and 7 show the closure member 41 in an open position exposing the intradermal needle 39, while FIGS. 6 and 8 show the closure member 41 in a closed position where the needle is protected.

The proximal end 38 of the adapter body 37 is enlarged and adapted for attachment to the collar 28. It has a pair of diametrally opposed, rectangular projections 42 for locking engagement in the respective apertures 32. A tapered surface 43 leads from each projection 42 distally to an annular flange 44. Each surface 43 has a profile corresponding to the profile 34 of the wall portions 33. The proximal end 38 is also provided with a conical recess 45 to accommodate the piercing member 20. The recess 45 leads to a narrow axial bore 46, which in turn leads to a narrow conical bore 47 in which the intradermal needle 39 is located. The needle 39 is held in the narrower proximal end 48 of the bore 47, while the slightly larger distal end 49 of the bore 47 opens into a conical recess 50 in the distal end 40 of the adapter body 37.

The distal end 40 of the adapter body 37 is also enlarged, and has the closure member 41 provided as a cap attached to one side of the end 40 by a living hinge 51. The distal end 40 has a flat end surface 52 from which the needle 39 projects by approximately 1.5 mm. The needle 39 is in the centre of the conical recess 50. An annular recess 53 is provided in the end surface 52, surrounding the conical recess 50 and spaced from it. The recess 53 allows space for the bleb to form following injection, and also provides for a snap engagement with the cap 41, so that the cap is held securely in the closed position.

The cap 41 has a flat surface 54 corresponding to, and for engagement with, the flat end surface 52 of the distal end 40. A conical recess 55 is provided at the centre of the surface 52 to correspond to conical recess 50, and in which the projecting end of the needle 39 is accommodated when the cap is in the closed position. An annular projection 56 is also provided, corresponding to the annular recess 53 of the distal end 40. The projection 56 has a hemispherical bulge 57 on its radially outer surface 58 which engages in a corresponding recess portion 59 of the recess 53 and provides the snap engagement of the cap 41 to the adapter body 37. The cap 41 has a projecting lug 60 opposite the hinge 51 to enable the cap 41 to be moved from the closed to the open position. A perceptible manual force is required to move the projection 56 out of the recess 53 to open the cap 41, and a similarly perceptible force is required to close it again.

Before use of the syringe 1, the piercing member 20 is protected by a transport cap 61 shown in FIGS. 9 and 10. The transport cap 61 comprises a cylindrical member 62 having an enlarged distal end 63 for ease of operation, and a proximal end 64 which fits over the piercing member 20 and is received within the annular space 35 between the collar 28 and the piercing member 20. The proximal end 64 has a closed recess 65 to protect the piercing member 20. A helical groove 66 is provided at the proximal end of the recess 65. The groove 66 allows gas to pass into the recess 65 for sterilising the piercing member 20. The transport cap 61 can therefore be assembled onto the syringe 1 and then the whole assembly of barrel, plunger and transport cap can be blister packed in a conventional manner and then sterilised using gas under pressure.

When the syringe 1 is to be used, it is removed from its packaging (not shown) by a health care worker. The transport cap 61 is removed to expose the piercing member 20. The health care worker then draws up the appropriate dose of an injectant, such as a vaccine, from a vial or ampoule (not shown). This is done by piercing a rubber septum closing the vial with the piercing point 27 so that the aperture 26 of the tapered bore 24 is in the injectant. The plunger 3 is then withdrawn to draw up the injectant through the tapered bore 24 into the distal part 5 of the barrel 2. The smaller diameter of the distal part 5 makes it easier to draw up the correct small volume (0.05 to 0.1 ml) required for intradermal injection. The distal part 5 of the barrel 2 may have external markings indicating the volume.

Once the syringe 1 is filled with the injectant, the adapter member 36 is attached to the connection member 21. The adapter member 36 is in its closed position, protecting the intradermal needle 39. The adapter member 36 can be attached in only two positions. The tapered wall portions 33 of the collar 28 indicate where these are, so that the projections 42 slide down the respective wall portions 33 until each projection 42 is received in an aperture 32. It will be appreciated that the projections 42 resiliently deform the collar 28 as they slide down the wall portions 33, and that the collar 28 resumes its original shape once the projections 42 reach the aperture 32. The profiles of the projections 42 and the wall portions 33 ensure that the projections 42 cannot be removed, and provide the locking engagement.

With the adapter member 36 attached, the syringe 1 is ready for injection. The health care worker applies a force, preferably a flick, with one hand to the lug 60 to move the cap 41 into the open position, exposing the needle 39. The hand will always be behind the needle, so that needlestick injury is avoided. The injection is given by placing the flat end surface 52 of the distal end 40 of the adapter body 37 on the surface of the skin, so that the intradermal needle 39 is inserted substantially perpendicularly into the skin. The flat surface 52 controls the penetration of the needle 39, so that the depth of insertion is limited to the 1.5 mm projection. The plunger 3 is then depressed fully to deliver the injectant from the barrel part 5 through the bore 24 in the piercing member 20, the aperture 26, the narrow axial bore 46 and the needle 39. The bleb that is formed is accommodated in the annular recess 53 and the conical recess 50.

After injection, the health care worker closes the cap 41, again with one hand always behind the needle, to reduce the risk of needlestick injury. The cap 41 is retained in the closed position by the snap engagement of the annular projection 56 in the annular recess 53. Although the cap 41 can be opened again, the adapter member 36 cannot be re-used, because it cannot be removed from the connection member 21 (at least, not without considerable damage). Further, the syringe 1 cannot be re-used, as full depression of the plunger 3 operates the auto-disable mechanism 11. On full depression, the radially outer ends of the arms of the cruciform member 15 engage in the groove 18 in the barrel 2. On withdrawal movement of the plunger 3 the arms flex, but then engage with the distal disc 14 to prevent them coming out of the groove 18. Any further attempt to withdraw the plunger 3 results in the central stem 17 breaking, rendering the plunger 3 unusable.

It will be appreciated that the syringe 1 is designed with a small dead space, because of the small diameters of the distal part 5 of the barrel, the tapered bore 24 of the piercing member 20 and the narrow bore 46. This ensures that the small doses of injectant can be given accurately, and with very little waste.

FIGS. 11 and 12 show a modification of the adapter member 36, and corresponding reference numerals have been applied to corresponding parts. In FIGS. 11 and 12 the modification consists of an axially-extending projection 68 depending from the lug 60 on the cap 41. The projection 68 is designed to grip the radially outer edge 66 of the distal end 40 of the adapter body 37. This provides extra security to keep the cap 41 in the closed position. Otherwise, the construction and operation of the adapter member 36 is the same as that of FIGS. 5 to 8.

FIGS. 13 and 14 show a further modification of the adapter member 36, and again corresponding reference numerals have been applied to corresponding parts. In FIGS. 13 and 14 the adapter body 37 is modified, such that it is of larger diameter at is proximal end 69, and the annular flange 44 is also of larger diameter. Raised portions 70 are provided on the larger diameter of the proximal end. The increased diameter and raised portions 70 make the adapter member 36 easier to grip when it is being attached to the syringe 1. Otherwise, the construction and operation of the adapter member 36 is the same as that of FIGS. 5 to 8. It will be appreciated that the reverse view shown in FIG. 14 shows the projections 42 and surfaces 43 in more detail.

FIGS. 15 and 16 show yet another modification of the adapter member 36, and corresponding reference numerals have been applied to corresponding parts. In FIGS. 15 and 16 the adapter body 37, cap 41 and hinge 51 are modified. The body 37 is modified to provide a straight rib 75 instead of a tapered surface 43 between the flange 44 and each projection 42, at the proximal end of the body 37. A further modification is to provide a straight cylindrical body portion 76 distally of the flange 44, leading to a tapered portion 77 containing the conical recess 45 adapted to accommodate the piercing member 20. The tapered portion 77 is provided with external axially-extending strengthening ribs 78. This construction reduces the amount of material required for the adapter body 37, while providing the necessary strength for the fluid pressure generated by an injection.

The distal end 40 of the adapter body 37 is also modified. The annular recess 53 is replaced by a shallow, narrow annular groove 79, so that the flat end surface 52 is enlarged. Radially inwardly of the groove 79 is a substantially frusto-conical projection 80, surrounding the conical recess 50, from which the needle 39 projects. The cap 41 is modified correspondingly, with a frusto-conical recess 81 instead of conical recess 55. The distance between the hinge 51 and the needle 39 is increased, for ease of use.

The syringe shown in FIGS. 17 to 24 is similar to that of FIGS. 1 to 4 and 7 to 10, and corresponding reference numerals have been applied to corresponding parts. In the syringe of FIGS. 1 to 4 and 7 to 10 the dead space is small, because of the small diameters of the distal part 5 of the barrel, the tapered bore 24 of the piercing member 20 and the narrow bore 46 of the adapter member 36. FIGS. 17 to 24 show a modification which reduces the dead space even further, to minimise waste of injectant.

Thus in FIGS. 17 to 24 the smaller diameter distal part 5 of the barrel 2 is increased in length, and the piercing member 20 is reduced in length, so that the syringe is no longer overall. In fact the portion 5 is extended up to the aperture 26, which is in direct communication with the barrel 2, so the tapered bore 24 is omitted. The external diameter of the reduced diameter portion 5 is increased in order to accommodate the increase in the internal diameter required by the plunger head 8. It will be appreciated that the plunger rod 7 is similarly extended, with its distal travel being limited by the distal end of the portion 5, adjacent the aperture 26. The distal end 85 of the plunger rod 7 is slightly tapered, and the elastomeric head 8 is a simple annulus located in a groove 86 adjacent the distal end 85.

The collar 28 is also modified, as best seen in FIG. 20. Each wall portion 33 leading from the open end 31 to the corresponding aperture 32 has a modified profile 34, with an inwardly-angled entry part 34a and an axially-extending part 34b leading to the aperture 32. This simple construction is possible because the external diameter of the barrel portion 5 surrounded by the collar 28 is less tapered than the part 20 of FIG. 1. This also enables the simpler construction of the adapter member 36 of FIGS. 15 and 16, with the straight ribs 75 instead of the tapered surfaces 43. It will be appreciated that the adapter member 36 of FIGS. 15 and 16 is shown in FIGS. 21 and 22.

The transport cap 61 is shown in FIGS. 23 and 24, and is also modified. The transport cap 61 is still a cylindrical member, but has no enlarged distal end. In fact the distal end 63 is tapered inwardly, and the external surface is provided with axial ribs 87 to enable it to be grasped securely.

Otherwise, the construction and operation of the syringe 1 of FIGS. 17 to 24 is the same as that of FIGS. 1 to 10, with the advantage that the waste of injectant is minimised by the reduced dead space.

It will be appreciated therefore that the syringes 1 in accordance with the first aspect of the invention provide a simple construction which is effective for giving intradermal injections, and are also easy and safe to use.

The adapter members 36 in accordance with the second aspect of the invention may be used in conjunction with the syringe 1 as illustrated. However, they may be modified for use with a standard syringe, having a standard luer connection so that it can be attached instead of a standard needle. While this may not provide the security features preventing re-use, it does provide a simple way of facilitating intradermal injection, perhaps with a pre-filled syringe.

1. A syringe comprises a barrel and a plunger sliding in the barrel and having a seal in sealing engagement with an internal surface of the barrel, the barrel having a piercing member with a piercing point and aperture at its distal end, the aperture being in communication with the distal end of the barrel, characterized in that the distal end of the barrel has a connection member to which an adapter member provided with an intradermal needle is adapted to be attached.

2. A syringe as claimed in claim 1, in which the piercing member and the connection member are formed integrally with the barrel.

3. A syringe as claimed in claim 1, in which the piercing point at the distal end of the piercing member is formed by an angled end of the piercing member, which also provides the aperture.

4. A syringe as claimed in claim 3, in which the aperture leads to a bore in the piercing member which provides communication with the distal end of the barrel.

5. A syringe as claimed in claim 4, in which the piercing member and the bore are tapered towards the point to assist with fluid flow.

6. A syringe as claimed in claim 3, in which the aperture leads directly to the distal end of the barrel.

7. A syringe as claimed in claim 1, in which the connection member comprises a collar member surrounding the piercing member or the distal end of the barrel.

8. A syringe as claimed in claim 7, in which an annular gap is provided between the piercing member or the distal end of the barrel and the collar member to accommodate the adapter member or a transport cap, which is assembled with the barrel to protect the piercing member for storage and transport until the syringe is to be used.

9. A syringe as claimed in claim 7, in which the collar member is arranged to lock the adapter member in place.

10. A syringe as claimed in claim 9, in which the adapter member comprises a body having at its proximal end a locking means for cooperation with the collar member, and carrying the intradermal needle at its distal end.

11. A syringe as claimed in claim 10, in which the distal end of the adapter member is arranged to accommodate a closure member to protect the intradermal needle before and after use.

12. A syringe as claimed in claim 1, in which the adapter body has an internal recess at its proximal end to accommodate the piercing member, the recess being tapered to correspond to the external profile of the piercing member.

13. A syringe as claimed in claim 12, in which the distal end of the internal recess leads to a narrow axial bore in which the intradermal needle is accommodated.

14. A syringe as claimed in claim 1, in which the distal end of the adapter body is substantially flat, with a conical recess through which the needle projects, and an annular recess spaced from and surrounding the conical recess.

15. A syringe as claimed in claim 11, in which the closure member for the distal end of the adapter body comprises a cap hinged to the adapter body, the proximal end of the cap having a conical recess to accommodate the projecting part of the needle, and an annular projection corresponding to the annular recess in the distal end of the adapter body.

16. A syringe as claimed in claim 9, in which the connection member has at least one aperture adapted to accommodate a corresponding projection in the adapter body.

17. A syringe as claimed in claim 16, in which a pair of opposing projections and apertures is provided, and each projection has a snap engagement with its corresponding aperture, the arrangement being a locking engagement such that the adapter body cannot be removed.

18. A syringe as claimed in claim 16, in which the connection member has a tapered surface leading to each aperture to guide the corresponding projection.

19. A syringe as claimed in claim 1, in which the barrel is of stepped outline, with a proximal portion of standard diameter and a distal portion of reduced diameter.

20. An intradermal injection adapter for a syringe, the syringe comprising a barrel and a plunger in slidable and sealing engagement therein, and the adapter comprising a body having an intradermal needle at a distal end, and a proximal end constructed and arranged for attachment to the barrel, the body also providing a fluid connection between the barrel and the intradermal needle.

21. A syringe as claimed in claim 20, in which the adapter has a standard luer connection to the syringe barrel.

22. A syringe as claimed in claim 20, in which the adapter has a locking connection to the barrel.

23. A syringe as claimed in claim 20, in which the adapter has a closure member able to be attached at its distal end to protect the intradermal needle before and after use.

24. A syringe as claimed in claim 23, in which the closure member is hinged to the adapter body.
